(19) United States
(12) Patent Application Publication
(10) Pub. No.: US 2007/0149887 A1
(43) Pub. Date: Jun. 28, 2007

(54) Portable electrocardiogram measurement device

(75) Inventors: Jin Sang Hwang, Suwon-si (KR); Kun Soo Shin, Seongnam-si (KR); Hyung Sok Yeo, Yongin-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/449,591

(22) Filed: Jun. 9, 2006

(30) Foreign Application Priority Data
Dec. 22, 2005 (KR) 10-2005-0127557

(51) Int. Cl. A61B 5/04

(52) U.S. Cl. 600/509; 600/393

(57) ABSTRACT

An ECG measurement device including: an ECG patch including a main body including a first side and a second side, the first side attached to the body of the subject; an electrode unit including at least three electrodes departed from each other at predetermined intervals and installed to the first side of the main body and conductive gel being applied to the periphery of each of the electrodes and receiving a pseudo ECG signal of the subject via the electrodes; and a first connector installed to the second side of the main body, corresponding to an installation position of each of the electrodes, electrically connected to the electrodes, physically attached and electrically connected to a predetermined controller to transmit the pseudo ECG signal to the controller; and an ECG measurement controller including a second connector physically attached and electrically connected to a first connector of the ECG measurement patch and receiving a pseudo ECG signal from the ECG measurement patch; an A/D converter converting the pseudo ECG signal into a digital signal; and a data transmission unit transmitting the pseudo ECG signal converted into the digital signal to a predetermined memory or a pseudo ECG signal analysis apparatus connected via wired/wireless network.

### CROSS-REFERENCE TO RELATED APPLICATIONS

This application claims the benefit of Korean Patent Application No. 10-2005-127557, filed on Dec. 22, 2005, in the Korean Intellectual Property Office, the disclosure of which is incorporated herein in its entity by reference.

### BACKGROUND OF THE INVENTION

1. Field of the Invention

The present invention relates to a portable electrocardiogram (ECG) measurement device, and more particularly, to a portable ECG measurement device formed of an ECG measurement patch including at least three electrodes and attached to a body of a subject to receive a pseudo ECG signal of the subject via the electrodes and an ECG measurement controller physically attached and electrically connected to the ECG measurement patch via a predetermined connector, receiving and converting the pseudo ECG signal into a digital signal, and transmitting the digital signal to a predetermined memory or a pseudo ECG signal analysis apparatus connected via a wired/wireless network.

2. Description of the Related Art

Ubiquitous technology indicates an information communication environment in which a user is unaware of a network or a computer and whose position is irrelevant while the user freely accesses the network. If Ubiquitous technology is generally used, anyone may freely use information technology not only in a house or in a vehicle but also on the top of a mountain. Also, since the general use of Ubiquitous technology increases the number of users of computers connected to a network, the information technology industry may be also expanded to a size and range in relation to the number of the users. Due to merits of not only portability and convenience, as described above, but also accessing a network in which time and position are irrelevant, technologies associated with a Ubiquitous technological system are increasingly being developed in nations worldwide.

The described technologies associated with Ubiquitous technology may be applied in every field of human life, and currently, in particular, due to the well-being fad, a ubiquitous healthcare (U-healthcare) is in the spotlight as a notable technical field. The U-healthcare indicates a ubiquitous technology in which a chip or sensor associated with medical services is integrated into any part of human life, thereby naturally providing medical services to anyone at anytime and anywhere. According to the U-healthcare, medical treatments performed in only hospitals, such as all sorts of medical examinations, management of diseases, emergency care, and consulting with doctors, may be provided without visiting hospitals.

For example, in the case of a diabetic, a belt for managing blood sugar, which is equipped with a blood sugar management program, may be worn. A blood sugar sensor attached to the belt may frequently check the blood sugar of the diabetic and may compute the amount of insulin suitable for the diabetic. When the blood sugar of the diabetic is rapidly increased or decreased, blood sugar information may be provided to an attending physician via a wireless communication network, and the attending physician receiving the blood sugar information may write out an optimal prescription or take optimal action according to an emergency situation.

As an example of U-healthcare, currently, portable electrocardiogram (ECG) measurement devices are commercialized and used by users who are afflicted with heart diseases. Due to a characteristic of heart diseases occurring at any moment, portable ECG measurement devices capable of continuously being carried and measuring ECG to forewarn of a sudden heart attack may be devices of interest associated with U-healthcare.

An ECG measurement device senses a weakly generated ECG signal in an organism and acquires an ECG waveform for determining whether heart disease exists. Accordingly, in a portable ECG measurement device, the structure, shape, and material of an electrode sensing a weakly generated ECG signal in an organism has a great effect on performance and efficiency of the entire measurement system.

According to conventional technology, an ECG measurement device uses a conductive hydrogel adhesive which does not cause skin irritation while simultaneously attaching an electrode to the skin in order to prevent inconsistent ECG measurements caused by electric potential or impedance caused by unreliable contact of the skin and electrode. However, when the ECG measurement device is used for more than a certain period, the adhesive coagulates and its function notably deteriorates.

Also, since several electrodes are used for measuring an ECG signal, each of the electrodes is connected to the measurement device via a corresponding lead line. In this case, an error may occur in measuring the ECG signal because, due to the several lead lines, adhesion around the skin cannot be sufficiently maintained when a subject moves.

Also, due to its characteristic, the ECG must be measured via a plurality of electrodes and a size and disposition of the electrodes may be the most important factors for measuring a precise ECG signal. However, since most portable ECG measurement devices according to conventional technologies do not sufficiently consider the size and disposition of the electrodes, which are important as described above, and are intended to only minimize electrodes, only the heart rate is measured and a precise ECG signal cannot be measured.

To solve the problems of conventional technologies, a portable ECG measurement device, in which a user can easily attach electrodes to his or her body and can easily detach the electrodes from the body, is required. Further, imprecise measurements of ECG, caused by several lead lines, can be prevented, while an optimal electrode size and disposition of electrodes are maintained, thereby being easily portable and guaranteeing precise ECG measurement.

### SUMMARY OF THE INVENTION

The present invention provides an ECG measurement patch in which at least three electrodes are separated from each other at predetermined intervals and each of the electrodes is designed to have a predetermined diameter, thereby precisely receiving an ECG signal from a subject.

The present invention also provides an ECG measurement controller that is physically attached and electrically connected to the ECG measurement patch via a predetermined connector, receives the ECG signal from the ECG measurement patch, and records the ECG signal in a memory or transmits the ECG signal to a predetermined ECG signal analysis apparatus, thereby preventing noise of the ECG signal, caused by movement of the subject when a lead line is connected.

The present invention also provides an ECG measurement controller that determines whether the subject is afflicted with a heart disease by analyzing the ECG signal received from the ECG measurement patch and generates an alarm signal when the heart disease occurs to replay or display via a display so that the subject may prepare for a heart disease that can occur at anytime and anywhere.

The present invention also provides an ECG measurement device formed of the ECG measurement patch which is physically attached and electrically connected to the ECG measurement controller via a connector in a single body so that the subject may easily carry and measure an ECG signal at anytime and anywhere.

According to an aspect of the present invention, there is provided an electrocardiogram measurement patch attached to a body of a subject in an electrocardiogram measurement device, including a main body including a first side and a second side, the first side attached to the body of the subject, an electrode unit including at least three electrodes separated from each other at predetermined intervals and installed to the first side of the main body and conductive gel being applied to the periphery of each of the electrodes and receiving a pseudo electrocardiogram signal of the subject via the electrodes, and a first connector installed to the second side of the main body, corresponding to an installation position of each of the electrodes, electrically connected to the electrodes, physically attached and electrically connected to a predetermined controller to transmit the pseudo electrocardiogram signal to the controller.

The controller may include a second connector physically attached and electrically connected to the first connector of the electrocardiogram patch and receiving the pseudo electrocardiogram signal, an A/D converter converting the pseudo electrocardiogram signal into a digital signal, and a data transmission unit transmitting the pseudo electrocardiogram signal converted into the digital signal to a predetermined memory or a pseudo electrocardiogram signal analysis apparatus connected via wired/wireless network.

According to another aspect of the present invention, there is provided an electrocardiogram measurement device including an electrocardiogram measurement patch including a main body including a first side and a second side, the first side attached to the body of the subject, an electrode unit including at least three electrodes separated from each other at predetermined intervals and installed to the first side of the main body and conductive gel being applied to the periphery of each of the electrodes and receiving a pseudo electrocardiogram signal of the subject via the electrodes, and a first connector installed to the second side of the main body, corresponding to an installation position of each of the electrodes, electrically connected to the electrodes, physically attached and electrically connected to a predetermined controller to transmit the pseudo electrocardiogram signal to the controller, and an electrocardiogram measurement controller including a second connector physically attached and electrically connected to a first connector of the electrocardiogram measurement patch and receiving the pseudo electrocardiogram signal from the electrocardiogram measurement patch, an A/D converter converting the pseudo electrocardiogram signal into a digital signal, and a data transmission unit transmitting the pseudo electrocardiogram signal converted into the digital signal to a predetermined memory or a pseudo electrocardiogram signal analysis apparatus connected via a wired/wireless network.

Additional aspects and/or advantages of the invention will be set forth in part in the description which follows and, in part, will be apparent from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a diagram illustrating a connection structure of an ECG measurement patch and ECG measurement controller according to an embodiment of the present invention;

FIG. 2 is a diagram illustrating a structure of an ECG measurement patch according to an embodiment of the present invention;

FIG. 3 is a graph illustrating characteristics of a waveform of a standard ECG signal;

FIG. 4 is a diagram illustrating a result of an experiment of measuring a value of a peak of an R wave of pseudo ECG signals respectively received, from a subject, by varying an interval between electrodes, according to an embodiment of the present invention;

FIG. 5 is a diagram illustrating a result of an experiment of measuring a value of a peak of an R wave of the pseudo ECG signals respectively received, from the subject, by varying a diameter of the electrodes, according to an embodiment of the present invention; and

FIG. 6 is a block diagram illustrating a configuration of an ECG measurement controller according to an embodiment of the present invention.

### DETAILED DESCRIPTION OF THE PREFERRED EMBODIMENTS

Reference will now be made in detail to the embodiments of the present invention, examples of which are illustrated in the accompanying drawings, wherein like reference numerals refer to the like elements throughout. The embodiments are described below to explain the present invention by referring to the figures.

The notation “ECG” frequently referred to in the present specification is an abbreviation of electrocardiogram. Excitement of the myocardium occurring in venous sinus advancing toward the heart atrium/cardiac ventricle may be illustrated in a graph of a current caused by the action of a heart, acquired by inducing an ammeter at two random points in a human body. The ECG is acquired by the described method and the ECG may be used as very important data for not only diagnoses of heart diseases but also diagnoses of several arrhythmia or electrolyte abnormality in addition to coronary artery diseases such as angina pectoris or myocardial infarction, and inspection and analysis of a heat during its operation of whether an abnormality exists.

An ECG measurement device according to one embodiment of the present invention may apply an ECG measurement method according to a standard limb lead including a first lead inducing an ammeter from both hands, a second lead inducing the ammeter from a right hand and a left foot, and a third lead inducing the ammeter. In addition, not only an ECG measurement method according to a unipolar lead or a chest lead but also all ECG measurement methods generally executed may be applied.

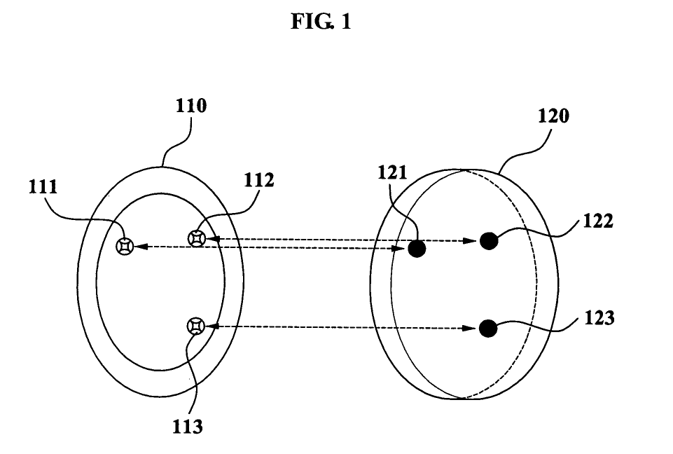

FIG. 1 is a diagram illustrating a connection structure of an ECG measurement patch and ECG measurement controller according to an embodiment of the present invention.

The ECG measurement device according to the present embodiment includes an ECG measurement patch 110 and an ECG measurement controller 120. A same number of connectors may be installed in the ECG measurement patch 110 and ECG measurement controller 120, respectively. The number of the connectors may be determined to be the same as a number of electrodes installed in the ECG measurement patch 110. In the present example illustrated in FIG. 1, the number of the connectors is three.

There may be installed first connectors 111, 112, and 113 on one side of the ECG measurement patch 110. Also, there may be installed second connectors 121, 122, and 123 on one side of the ECG measurement controller 120.

The ECG measurement patch 110 and the ECG measurement controller 120 may be attached to each other by coupling of the first connectors 111, 112, and 113, and the second connectors 121, 122, and 123. Namely, the first connector 111 is coupled with the second connector 121, the first connector 112 is coupled with the second connector 122, and the first connector 113 is coupled with the second connector 123, thereby attaching the ECG measurement patch 110 to the ECG measurement controller 120.

For this, the first connectors 111, 112, and 113 and the second connectors 121, 122, and 123 may be embodied in the shape of a pair of hook switches capable of being coupled with each other. Also, the first connectors 111, 112, and 113 and the second connectors 121, 122, and 123 may be embodied as conductors capable of being electrically connected. Accordingly, the ECG measurement patch 110 and the ECG measurement controller 120 may be not only physically attached to each other but also electrically connected to each other.

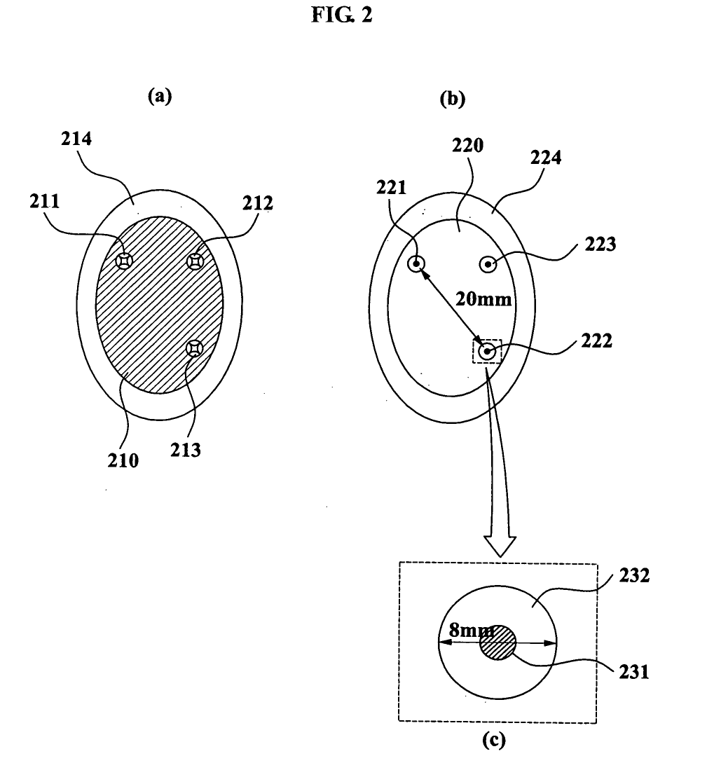

The ECG measurement patch 110 may be embodied to have two sides. The first connectors 111, 112, and 113 may be installed on the one side as described above, and electrodes may be installed on the other side. This will be described in detail with reference to FIG. 2.

FIG. 2 is a diagram illustrating a structure of an ECG measurement patch according to an embodiment of the present invention.

The ECG measurement patch according to the present embodiment includes main bodies 210 and 220, electrode units 221 through 223, and connectors 211 through 213 and may further include auxiliary adhesive units 214 and 224.

The main bodies 210 and 220 of the ECG measurement patch may include a first side and a second side. A predetermined adhesive may be applied to the first side to attach the ECG measurement patch to the body of a subject. The auxiliary adhesive units 214 and 224 may also be adhered to the second side. The auxiliary adhesive units 214 and 224 may be embodied to be larger than the main bodies 210 and 220. The ECG measurement patch may be embodied to be much more attached to the body of the subject by applying the adhesive to one side of the auxiliary adhesive unit 224.

In FIG. 2, (a) illustrates the second side of the ECG measurement patch and (b) illustrates the first side of the ECG measurement patch.

As illustrated in (a) of FIG. 2, the three connectors 211 through 213 may be installed on the second side of the main body 210. As described with reference to FIG. 1, the connectors 211, 212, and 213 may be physically attached and electrically connected to the ECG measurement controller, respectively.

Also, the connectors 211 through 213 may be installed in positions on the second side, which respectively correspond to the electrode units 221 through 223 installed on the first side of the main body 210. Namely, the connectors 211 through 213 may face the electrode units 221 through 223, and are interposed between the main bodies 210 and 220.

On the first side of the main body 220 of the ECG measurement patch illustrated in (b) of FIG. 2, the electrode units 221 through 223 are installed, which include three electrodes installed and separated from each other at predetermined intervals, respectively. Conductive gel is applied to the periphery of each of the electrodes, and each of the electrodes receives a pseudo ECG signal of the subject.

Namely, the electrode units 221 through 223 may include electrodes and conductive gel. The electrodes may receive a pseudo ECG signal from the subject when the first side is in contact with the body of the subject. The electrodes may be embodied to have characteristics identical with electrodes used in a general ECG measurement device.

The conductive gel may be applied to the periphery of the electrodes. Since the conductive gel has a characteristic of a conductor, the conductive gel may be applied as a means for enlarging an area of the electrode. That is, since the conductive gel is also in contact with the body of the subject and senses the pseudo ECG signal, the sensing area of the pseudo ECG signal received from the subject to the electrode may be enlarged.

Accordingly, by establishing a size of the area for applying the conductive gel, the pseudo ECG signal sensing area of the electrode may be controlled. However, in order to sense the pseudo ECG signal, the adhesive applied to the main body 220 may not be applied to the conductive gel or the electrode.

The connectors 211 through 213 and the electrode units 221 through 223 may be installed to be electrically connected to each other. Namely, in order to transmit the subject's pseudo ECG signal inputted via the electrode units 221 through 223 to the ECG measurement controller via the connectors 211 through 213, the connectors 211 through 213 and the electrode unit 221 through 223 may be embodied to be electrically connected to each other.

According to an embodiment of the present invention, a first electrode unit 221, a second electrode unit 222, and a third electrode 223 may be installed on the first side of the main body 220. The first electrode unit 221 may be embodied as a positive electrode, the second electrode unit 222 may be embodied as a negative electrode, and the third electrode unit 223 may be embodied as a ground electrode.

Also, each of the electrode units is separated from each other at predetermined intervals, respectively, on the first side of the main body 220. For example, as illustrated in (b) of FIG. 2, the first electrode unit 221 may be installed to be separated from the second electrode unit 222 by 20 mm.

Also, the electrode unit may be designed to have a predetermined diameter. For example, as illustrated in (c) of FIG. 2, the electrode unit may have a diameter of 8 mm. The diameter may be embodied by including conductive gel 232 applied to the periphery of electrode 231.

As described above, the electrode unit is embodied to have the predetermined installation intervals and diameter to more efficiently sense the pseudo ECG signal from the subject. In a general ECG measurement device, due to medical reasons, electrodes may be attached to parts of the body of the subject, such as right arm, left arm, and right leg, and may receive a standard ECG signal from the subject.

However, since the ECG measurement device according to the present embodiment is embodied as a portable device in which electrodes are installed on one patch, there is a difficulty in sensing the standard ECG signal using the described general method. Accordingly, the pseudo ECG signal is received from the subject via the three electrodes installed on the ECG measurement patch, and whether a heart disease of the subject occurs may be determined according to correlation between the pseudo ECG signal and the standard ECG signal.

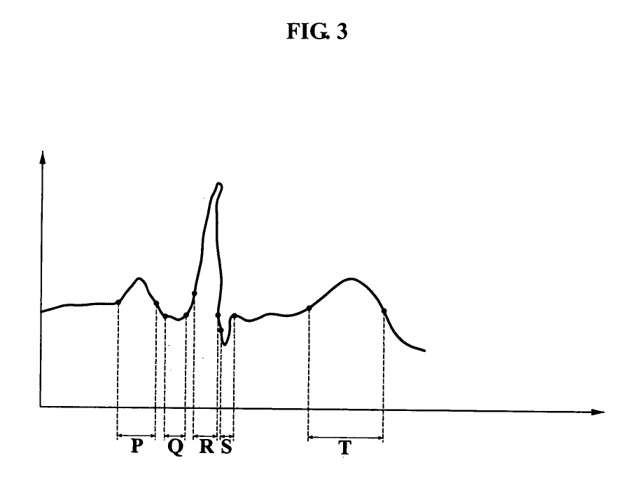

The operation of receiving a precise pseudo ECG signal via the three electrodes installed in the ECG measurement patch is very important. To input the precise pseudo ECG signal, an optimal electrode disposition interval and the electrode diameter have to be previously determined. This can be determined by a predetermined experiment, which will be described in detail with reference to FIGS. 3 through 5.

FIG. 3 is a graph illustrating characteristics of a waveform of the standard ECG signal. A heart is a pump circulating blood throughout the whole body and regularly repeats contraction and expansion without rest. Pumping of the heart is performed by extraction of the myocardium. A weak electrical signal occurs whenever the heart beats, thereby causing a flow of current through the body. Distribution of electric potential is generated on the surface of the body by the current. The ECG, which is a change of electric potential in the heart caused by activity of the heart, is induced from a suitable part of the surface of the body of a subject and is amplified and recorded by a certain method.

The waveform of the ECG, which reflects the steps of electrical activation of the heart basically includes P, Q, R, S, and T waves, as shown in FIG. 3. The P wave is generated during heart atrium depolarization, the group of Q, R, and S waves is generated during cardiac ventricle depolarization, and T wave is generated during cardiac ventricle repolarization.

The depolarization of the heart atrium starts in the vicinity of sinuatrial node and crosses the heart atrium from right to left. Accordingly, a front part of the P wave indicates depolarization of right atrium, and a rear part of the P wave indicates depolarization of left atrium. Normally, the P wave is generated for diastole of the cardiac ventricle.

The group of Q, R, and S waves reflects the depolarization of the cardiac ventricle. The depolarization of the cardiac ventricle starts with a left part of the interventricular septum in the vicinity of the AV junction and crosses the interventricular septum from left to right. Accordingly, the Q wave indicates depolarization of the interventricular septum, and other parts of the group of Q, R, and S waves indicate depolarization of left/right ventricles, which occurs synchronously. The R wave is defined to be a first upturn wave that is recorded. The Q wave is defined to be a downturn wave recorded before the R wave, and the S wave is defined to be a downturn wave recorded after the R wave. An abnormal group of Q, R, and S waves may be seen in a conduction defect in the cardiac ventricle, for example, bundle branch block, and preexcitation syndrome as an example of an atrioventricular conduction disturbance.

A normal T wave indicates normal repolarization of the cardiac ventricle. The normal repolarization starts with the surface of epicardium of the cardiac ventricle and is progressed toward endocardium via a ventricular wall. The T wave is generated during the end of systole of the cardiac ventricle.

It may be possible to recognize an abnormality of the P wave, the group of Q, R, and S waves, and T wave by such factors as a duration of each wave, an interval between adjacent waves, a segment for connecting with an adjacent wave, an amplitude of a wave, and sharpness of a wave and may be analyzed to determine whether such values are in a normal range.

Specifically, the amplitude of the wave is the most important factor of the factors. A peak value of the R wave, whose amplitude is greatest, may be determined to be an important factor. By using this approach, the correlation between the pseudo ECG signal and the standard ECG signal may be deduced by comparison between the peak value of the R wave of the pseudo ECG signal measured by the ECG measurement device according to the present embodiment, and the peak value of the R wave of the standard ECG signal.

Accordingly, to precisely measure the peak value of the R wave of the pseudo ECG signal, the interval between each of the electrodes installed on the ECG measurement patch and the diameter of each of the electrodes are determined to be various values and the peak values of the R wave of the pseudo ECG signal measured from the subject are compared with each other, thereby computing the optimal interval between the electrodes and the optimal diameter of the electrode.

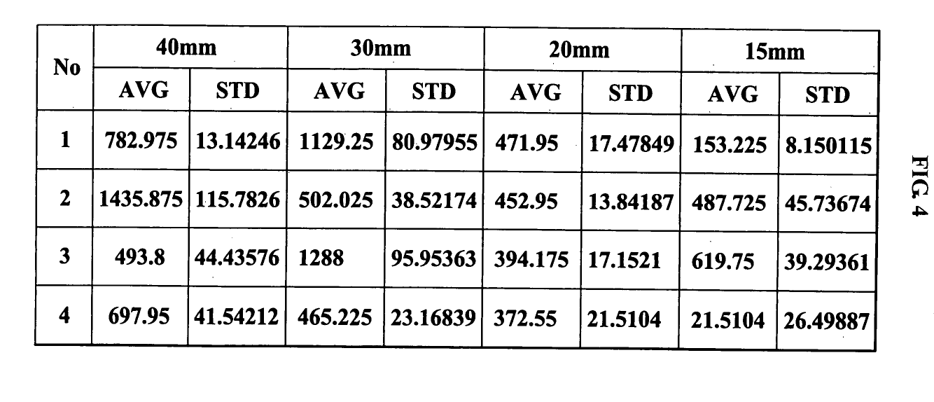

FIG. 4 is a diagram illustrating a result of an experiment of measuring a peak value of an R wave of pseudo ECG signals received, respectively, from a subject, which are received while varying an interval between electrodes, according to an embodiment of the present invention.

According to the result of the experiment illustrated in FIG. 4, it may be seen that an average and standard deviation of the peak value of the R wave of the pseudo ECG signal measured in the case the interval between the electrodes is 20 mm are at uniform levels. Accordingly, as a result of the experiment, the optimal interval between the electrodes may be determined to be 20 mm.

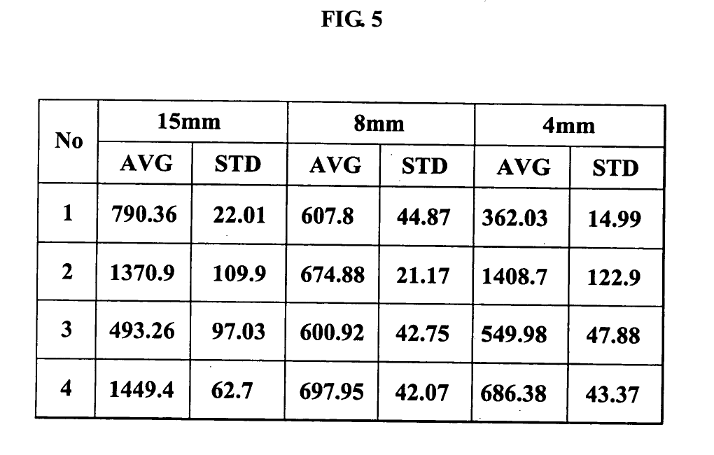

FIG. 5 is a diagram illustrating a result of an experiment of measuring the peak value of the R wave of the pseudo ECG signals received, respectively, from the subject, which are received while varying a diameter of the electrodes, according to an embodiment of the present invention.

Referring to FIG. 5, it may be seen that an average and standard deviation of the peak value of the R wave of a pseudo ECG signal measured in the case the diameter of the electrode is 8 mm are at uniform levels. Accordingly, as a result of the experiment, the optimal diameter of the electrode may be determined to be 8 mm.

The optimal values of the interval between the electrodes installed on the ECG measurement patch and the diameter of the electrode are described with reference to FIGS. 3 through 5. According to one embodiment of the present invention, an interval between the electrodes may be determined to be an interval between the positive electrode and the negative electrode, as illustrated in FIG. 2. Also, the diameter of the electrode may be determined to be a diameter of the electrode unit including the conductive gel.

Also, the optimal values of the interval between the electrodes and the diameter of the electrode are just an example of results of experiments performed according to an embodiment of the present invention. Various optimal values of the interval between the electrodes and the diameter of the electrode may be acquired by a variety of additional experiments.

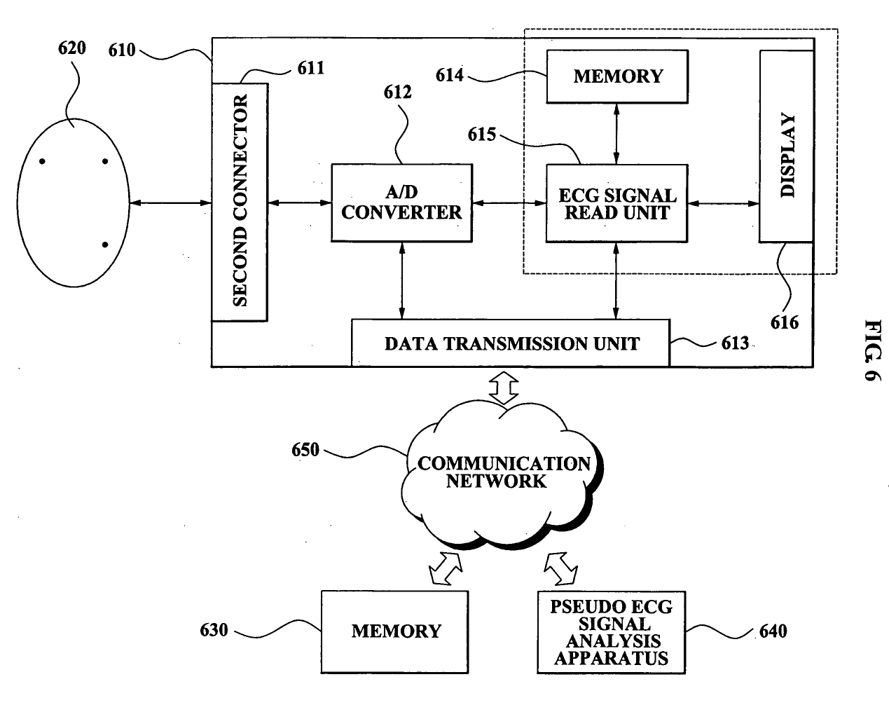

FIG. 6 is a block diagram illustrating a configuration of an ECG measurement controller 610 according to an embodiment of the present invention.

The ECG measurement controller 610 includes a second connector 611, an A/D converter 612, and a data transmission unit 613. Also, the ECG measurement controller 610 may further include a memory 614, an ECG signal read unit 615, and a display 616.

The second connector 611 is physically attached to a first connector of an ECG measurement patch 620, electrically connected to the first connector, and receives a pseudo ECG signal from the ECG measurement patch 620. The second connector 611 may be installed on a side where the ECG measurement controller 610 is coupled with the ECG measurement patch 620 or may be installed on a position in the side, corresponding to the first connector of the ECG measurement patch 620. The second connector 611 and the first connector may be embodied as a predetermined pair of hook switches to be coupled.

The A/D converter 612 converts the pseudo ECG signal into a digital signal. Namely, the A/D converter 612 may amplify the pseudo ECG signal, which is an analog signal received from the first connector of the ECG measurement patch via the second connector 611, to convert the analog pseudo ECG signal into the digital signal. The embodiment of the A/D converter 612 may include a predetermined differential amplification circuit and an A/D conversion circuit.

The data transmission unit 613 transmits the pseudo ECG signal converted into the digital signal to a predetermined memory 630 or an ECG signal analysis apparatus 640 connected via a communication network 650 which may be a wired/wireless network. In the case the memory 614 is additionally formed outside the ECG measurement controller 610, the data transmission unit 613 may transmit the pseudo ECG signal converted into the digital signal to the memory 630. The transmitted pseudo ECG signal is recorded in the memory 630.

According to an embodiment of the present invention, a user, for example, a doctor, may diagnose whether the subject has a heart disease from the pseudo ECG signal recorded in the memory 630. Namely, the user connects the memory 630 to a predetermined analysis apparatus such as a PC and reads the pseudo ECG signal displayed via the analysis apparatus, thereby diagnosing whether the subject has a heart disease.

In this case, the subject may carry an ECG measurement device including the ECG measurement patch 620 and the ECG measurement controller 610 as a necklace and may continuously measure and record ECG of the subject in the memory 630. In this case, the memory 630 may be embodied to be placed into a pants pocket or to be worn on the waist. Accordingly, the subject measures and records his or her ECG in the memory 630 at anytime and anywhere and a doctor reads the usual ECG of the subject, recorded in the memory 630, when the subject visits a hospital, thereby acquiring an effect of precisely diagnosing a heart disease of the subject.

For the described operation, the memory 630 may include a connection unit connected to the analysis apparatus, such as a USB port. Also, for the operation of transmitting and receiving the pseudo ECG signal, the data transmission unit 613, the memory 630, and the pseudo ECG signal analysis apparatus 640 may include a predetermined communication module.

The communication module may include a short-range communication module for performing short-range communication, such as with Wireless LAN (WLAN), Bluetooth, Ultra-wideband (UWB), Infrared Data Association (IrDA), Home Phoneline Networking Alliance (HPNA), Shared Wireless Access Protocol (SWAP), and Institute of Electrical and Electronics Engineers standard 1394 (IEEE1394). Also, the communication module may support at least one of public switched telephone network (PSTN), Code Division Multiple Access (CDMA), Wideband CDMA (WCDMA), all IP, Global System for Mobile Communication (GSM), General Packet Radio Service (GPRS), and existing access methods related to mobile communication and may be embodied to support at least one protocol of call control protocols for accessing Voice over Internet Protocol (VoIP) call such as H.323, Message Gateway Control Protocol (MGCP), Session Initiation Protocol (SIP), or Megaco.

Also, the communication network 650 supporting a wired/wireless network may be embodied to be a mobile communication network supporting at least one of CDMA, WCDMA, ALL IP, GSM, GPRS access methods, and any other existing access methods related to mobile communication. The communication network 650 may be embodied as a wired or wireless Internet and may be embodied to include a communication network that will be provided in the future, such as mobile Internet and VoIP. Also, the communication network 650 may be embodied including the short-range communication network such as WLAN, Bluetooth, UWB, IrDA, HPNA, SWAP, and IEEE1394.

According to another embodiment of the present invention, the ECG measurement controller 610 may further include the memory 614, the ECG signal read unit 615, and the display 616. Namely, the pseudo ECG signal converted into the digital signal via the A/D converter 612 is not transmitted to the memory 630 or the pseudo ECG signal analysis apparatus 640 by the data transmission unit 613. The pseudo ECG signal may be recorded in the memory 614, or the ECG signal read unit 615 may diagnose whether the subject has a heart disease, from the pseudo ECG signal.

A predetermined ECG signal algorithm may be recorded and maintained in the memory 614. The ECG signal algorithm may be embodied to be a predetermined program to diagnose whether the subject has a heart disease, from the pseudo ECG signal. Accordingly, the ECG signal read unit 615 may diagnose whether the subject has a heart disease, from the pseudo ECG signal via the ECG signal algorithm.

The ECG read unit 615 may display the pseudo ECG signal via the display 616 to the subject or the user. Also, in the case it is determined that, as a result of reading the pseudo ECG signal, a heart disease of the subject occurs, the ECG signal read unit 615 may generate a predetermined alarm signal and may replay or display the alarm via the display to the subject or the user.

For example, the ECG signal read unit may diagnose arrhythmia of the heart of the subject by using the ECG signal algorithm. To diagnose the arrhythmia, the ECG signal read unit 615 detects an R-peak of the pseudo ECG signal and computes an interval of R-peaks, namely, an R-R interval.

After computing the R-R interval, the ECG signal read unit 615 compares the computed R-R interval with an R-R interval previously determined by the ECG signal algorithm. As a result of the comparison, when the computed R-R interval has discrepancies with the previously determined R-R interval, it may be determined that a heart arrhythmia of the subject occurs.

As described above, according to another embodiment of the present invention, the ECG measurement controller 610 may include elements capable of recording and displaying the pseudo ECG signal and diagnosing whether a heart disease of the subject occurs.

Accordingly, the subject carries the ECG measurement device in which the ECG measurement patch and the ECG measurement controller are integrated, measures the subject's ECG at anytime and anywhere, and analyzes whether a heart disease of the subject occurs, thereby being desirably applied to the U-healthcare. Also, the pseudo ECG signal is transmitted to an external terminal or server such as the pseudo ECG signal analysis apparatus via a communication module, thereby enabling rapid request for help when an emergency situation occurs.

Accordingly, there is provided an ECG measurement patch in which at least three electrodes are separated from each other at predetermined intervals and each of the electrodes is designed to have a predetermined diameter, thereby receiving an ECG signal from a subject.

Accordingly, there is provided an ECG measurement controller that is physically attached and electrically connected to the ECG measurement patch via a predetermined connector, receives the ECG signal from the ECG measurement patch, and records the ECG signal in a memory or transmits the ECG signal to a predetermined ECG signal analysis apparatus, thereby preventing noise of the ECG signal, caused by movement of the subject when a lead line is connected.

Accordingly, there is provided an ECG measurement controller that determines whether the subject is afflicted with a heart disease by analyzing the ECG signal received from the ECG measurement patch and generates an alarm signal when the heart disease occurs to replay or display via a display so that the subject may prepare for a heart disease that can occur at anytime and anywhere.

Accordingly, there is provided an ECG measurement device formed of the ECG measurement patch which is physically attached and electrically connected to the ECG measurement controller via a connector in a single body so that the subject may easily carry and measure an ECG signal at anytime and anywhere.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. An electrocardiogram measurement patch attached to a body of a subject in an electrocardiogram measurement device, comprising:
a main body including a first side and a second side, the first side attached to the body of the subject;
an electrode unit including at least three electrodes separated from each other at predetermined intervals and installed to the first side of the main body and conductive gel being applied to the periphery of each of the electrodes and receiving a pseudo electrocardiogram signal of the subject via the electrodes; and
a first connector installed to the second side of the main body, corresponding to an installation position of each of the electrodes, electrically connected to the electrodes, physically attached and electrically connected to a predetermined controller to transmit the pseudo electrocardiogram signal to the controller,
wherein the controller includes a second connector physically attached and electrically connected to the first connector of the electrocardiogram patch and receiving the pseudo electrocardiogram signal, an A/D converter converting the pseudo electrocardiogram signal into a digital signal, and a data transmission unit transmitting the converted digital pseudo electrocardiogram signal to a predetermined memory or to a pseudo electrocardiogram signal analysis apparatus connected via a wired/wireless network.

2. The patch of claim 1, wherein:
the electrode unit includes a positive electrode, a negative electrode, and a ground electrode;
a separation distance between the positive electrode and the negative electrode is at least 20 mm; and
each of the electrodes has at least an 8 mm diameter.

3. An electrocardiogram measurement controller attached to an electrocardiogram measurement patch in an electrocardiogram measurement device, comprising:
a second connector physically attached and electrically connected to a first connector of the electrocardiogram measurement patch and receiving a pseudo electrocardiogram signal from the electrocardiogram measurement patch;
an A/D converter converting the pseudo electrocardiogram signal into a digital signal; and
a data transmission unit transmitting the pseudo electrocardiogram signal converted into the digital signal to a predetermined memory or a pseudo electrocardiogram signal analysis apparatus connected via a wired/wireless network,
wherein the electrocardiogram measurement patch includes a main body including
a first side and a second side, the first side attached to the body of the subject;
an electrode unit including at least three electrodes separated from each other at predetermined intervals and installed to the first side of the main body and conductive gel being applied to the periphery-of each of the electrodes and receiving a pseudo electrocardiogram signal of the subject via the electrodes; and
a first connector installed to the second side of the main body, corresponding to an installation position of each of the electrodes, the first connector electrically connected to the electrodes, physically attached and electrically connected to a predetermined controller to transmit the pseudo electrocardiogram signal to the controller.

4. The controller of claim 3, wherein the memory maintains a predetermined electrocardiogram signal algorithm, further comprising an electrocardiogram signal read unit diagnosing whether a heart disease of the subject occurs from the pseudo electrocardiogram signal via the electrocardiogram signal algorithm, wherein the electrocardiogram measurement controller includes the memory and the pseudo electrocardiogram signal converted into the digital signal is recorded in the memory.

5. The controller of claim 4, further comprising a display for displaying the pseudo electrocardiogram signal to the subject,
wherein when it is determined that the heart disease of the subject occurs, the electrocardiogram signal read unit generates a predetermined alarm signal and replays or displays the alarm signal to the subject via the display.

6. The controller of claim 4, wherein the electrocardiogram signal read unit detects an R-peak of the pseudo electrocardiogram signal, computes the R-peak intervals, and determines the heart disease to be an arrhythmia in the case the computed intervals of the R-peak is not identical with predetermined intervals.

7. An electrocardiogram measurement device, comprising:
an electrocardiogram measurement patch including a main body including a first side and a second side, the first side attached to the body of a subject;
an electrode unit including at least three electrodes separated from each other at predetermined intervals and installed to the first side of the main body and conductive gel being applied to the periphery of each of the electrodes and receiving a pseudo electrocardiogram signal of the subject via the electrodes;
a first connector installed to the second side of the main body, corresponding to an installation position of each of the electrodes, the first connector electrically connected to the electrodes, physically attached and electrically connected to a predetermined controller to transmit the pseudo electrocardiogram signal to the controller;
an electrocardiogram measurement controller including a second connector physically attached and electrically connected to the first connector of the electrocardiogram measurement patch and receiving the pseudo electrocardiogram signal from the electrocardiogram measurement patch;
an A/D converter converting the pseudo electrocardiogram signal into a digital signal; and
a data transmission unit transmitting the converted digital pseudo electrocardiogram signal to a predetermined memory or a pseudo electrocardiogram signal analysis apparatus connected via wired/wireless network.

8. A main body attached to a body of a subject, comprising:
an electrode unit including a plurality of electrodes separated from each other at predetermined intervals and installed to the main body and conductive gel; and
a connector installed to the main body, the connector electrically connected to the electrodes, and connected to a predetermined controller to transmit a pseudo electrocardiogram signal to the controller,
wherein the controller includes an A/D converter converting the pseudo electrocardiogram signal into a digital signal, and a data transmission unit transmitting the converted digital pseudo electrocardiogram signal to at least one external device.

9. An electrocardiogram measurement apparatus, comprising:
a controller unit comprising a second connector, the second connector configured to be connected to a first connector of a electrocardiogram patch in order to receive a pseudo electrocardiogram signal;
an A/D converter unit to convert the received pseudo electrocardiogram signal into a digital signal; and
a transmission unit to transmit the converted digital pseudo electrocardiogram signal to a predetermined memory and/or to a pseudo electrocardiogram signal analysis apparatus connected via a wired/wireless network.

10. An electrocardiogram (ECG) measurement apparatus, comprising:
an ECG measurement patch comprising a plurality of at least three electrodes;
a body of a subject attached to the ECG measurement patch to receive pseudo ECG signals of the subject via the plurality of electrodes;
an A/D converter to convert the received pseudo ECG signal into a digital signal;
a transmitter to transmit the converted digital signal to a predetermined memory and/or a pseudo ECG signal analysis apparatus connected via a wired/wireless network.
